(19) United States
(12) Patent Application Publication
(10) Pub. No.: US 2023/0301889 A1
(43) Pub. Date: Sep. 28, 2023

(54) METHOD OF PREVENTING OR REDUCING HAIR FALL

(71) Applicant: Conopco, Inc., d/b/a UNILEVER, Englewood Cliffs, NJ (US)

(72) Inventors: Jiayin GU, Shanghai (CN); Xiaoying BIAN, Shanghai (CN); Yingjie LI, Shanghai (CN); Binzuo LIU, Shanghai (CN); Shujun CAI, Shanghai (CN)

(73) Assignee: Conopco, Inc., d/b/a UNILEVER, Englewood Cliffs, NJ (US)

(21) Appl. No.: 18/022,799

(22) PCT Filed: Aug. 24, 2021
(86) PCT No.: PCT/EP2021/073325

§ 371 Date: Feb. 23, 2023

(30) Foreign Application Priority Data
Sep. 14, 2020 (WO) PCT/CN2020/115051
Oct. 14, 2020 (EP) 20201785.1

(51) Int. Cl. A61K 8/49; A61K 8/27; A61K 8/892; A61Q 5/02

(52) U.S. Cl. CPC A61K 8/4926; A61K 8/27; A61K 8/892; A61Q 5/02; A61K 2800/70

(57) ABSTRACT

The invention provides the use of a hair care composition comprising piroctone olamine, a zinc salt and a silicone for preventing or reducing shedding of hair club fibres from hair follicles, and/or preventing or reducing hair breakage, thereby preventing or reducing hair fall.

### FIELD OF THE INVENTION

The present invention relates to use of hair care composition for preventing or reducing hair fall. In particular the present invention relates to use of hair care composition comprising piroctone olamine, a zinc salt and a silicone for preventing or reducing hair fall.

### BACKGROUND OF THE INVENTION

Loss of hair (hair fall) is a major concern for adults around the world. Thus there has been intensive scientific investigation into the causes of hair fall over many years.

The hair follicle is a regenerating biological system whose primary function is to produce a hair fibre. The hair growth cycle consists of phases of active regeneration (anagen), apoptotic involution (catagen), and relative proliferative quiescence (telogen), which occur continuously throughout the follicle lifetime. Towards the end of catagen and at the beginning of telogen, a completely keratinized hair shaft with a characteristic depigmented and club-like base, termed the “hair club fibre”, is held firmly by a specialized junction complex at the base of the bulge (a group of slow-cycling keratinocytes located below the sebaceous gland).

In humans, the hair club fibre is not usually retained through subsequent anagens, but shed from the follicle in a distinct and independent phase termed “exogen”. Hair shedding is thus a natural consequence of the human hair cycle. However, the amount of hair that is shed depends on several internal and external factors, and the conscious perception of hair shedding can vary by individual. Premature or excessive hair shedding can be a preoccupation for many people, not only for those with a recognised hair loss disorder.

There is a continuing need for cosmetically acceptable treatments which are capable of preventing or reducing hair fall, especially preventing or reducing hair shedding through targeting of processes involved in exogen.

### SUMMARY OF THE INVENTION

The invention provides the use of a hair care composition comprising piroctone olamine, a zinc salt and a silicone for preventing or reducing shedding of hair club fibres from hair follicles, and/or preventing or reducing hair breakage, thereby preventing or reducing hair fall. This aspect may alternatively be described in any of the following ways:
A method of preventing or reducing hair fall in an individual in need thereof, comprising topically applying a hair care composition comprising piroctone olamine, a zinc salt and a silicone to the hair and/or scalp of the individual.
A hair care composition comprising piroctone olamine, a zinc salt and a silicone for use as a medicament, preferably for use as a medicament in the treatment or prevention of hair fall.
Use of a hair care composition comprising piroctone olamine, a zinc salt and a silicone in the manufacture of a medicament for the treatment or prevention of hair fall.

The invention also provides a method of preventing or reducing hair fall in an individual in need thereof, comprising topically applying a hair care composition comprising piroctone, a zinc salt and a silicone to the hair and/or scalp of the an individual, in an amount effective to reduce shedding of hair club fibres from hair follicles of the individual, and/or reduce hair breakage of the individual.

The invention further provides a hair care composition comprises piroctone olamine, a zinc salt and a silicone for use in preventing or reducing shedding of hair club fibres from hair follicles, and/or preventing or reducing hair breakage, thereby preventing or reducing hair fall.

All other aspects of the present invention will more readily become apparent upon considering the detailed description and examples which follow.

### DETAILED DESCRIPTION

For the avoidance of doubt, any feature of one aspect of the present invention may be utilized in any other aspect of the invention. The word “comprising” is intended to mean “including” but not necessarily “consisting of or “composed of.” In other words, the listed steps or options need not be exhaustive. It is noted that the examples given in the description below are intended to clarify the invention and are not intended to limit the invention to those examples per se. Similarly, all percentages are weight/weight percentages unless otherwise indicated. Except in the operating and comparative examples, or where otherwise explicitly indicated, all numbers in this description and claims indicating amounts of material or conditions of reaction, physical properties of materials and/or use are to be understood as modified by the word “about”. Numerical ranges expressed in the format “from x to y” are understood to include x and y. When for a specific feature multiple preferred ranges are described in the format “from x to y”, it is understood that all ranges combining the different endpoints are also contemplated. As used herein, the indefinite article “a” or “an” and its corresponding definite article “the” means at least one, or one or more, unless specified otherwise. The various features of the present invention referred to in individual sections above apply, as appropriate, to other sections mutatis mutandis. Consequently, features specified in one section may be combined with features specified in other sections as appropriate. Any section headings are added for convenience only and are not intended to limit the disclosure in any way.

“Hair care composition”, as used herein, is meant to include a composition for topical application to hair and/or scalp of mammals, especially humans. Such a composition may be generally classified as leave-on or rinse off, and includes any product applied to a human body for also improving appearance, cleansing, odor control or general aesthetics. The composition of the present invention can be in the form of a liquid, lotion, cream, foam, scrub, gel, or bar. Non-limiting examples of such compositions include leave-on hair lotions, creams, and rinse-off shampoos, conditioners, shower gels, or toilet bar.

“Hair fall”, as used herein, is meant to include the hair shedding from the hair follicle and hair loss due to the hair breakage.

The present invention provides the use of a hair care composition comprising piroctone olamine, a zinc salt and a silicone for preventing or reducing shedding of hair club fibres from hair follicles, and/or preventing or reducing hair breakage, thereby preventing or reducing hair fall. Preferably, the present invention provides for use of a hair care composition comprising piroctone olamine, a zinc salt and a silicone for preventing or reducing shedding of hair club fibres from hair follicles, thereby preventing or reducing hair hall. The use is preferably non-therapeutic in nature, more preferably cosmetic in nature.

Preferably, the present invention provides the use of a hair care composition for preventing or reducing shedding of hair club fibres from hair follicles, and/or preventing or reducing hair breakage, thereby preventing or reducing hair fall, wherein the hair care composition comprises: piroctone olamine, wherein the hair care composition provides a greater anti-hair fall benefit in comparison to a hair care composition that does not contain the piroctone olamine; a zinc salt; and a silicone. Anti-hair fall benefit includes preventing or reducing hair fall.

Preferably, the present invention provides the use of a hair care composition for preventing or reducing shedding of hair club fibres from hair follicles, and/or preventing or reducing hair breakage, thereby preventing or reducing hair fall, wherein the hair care composition comprises: piroctone olamine; a zinc salt; and a silicone; wherein the hair care composition provides a greater anti-hair fall benefit in comparison to a hair care composition that does not contain the combination of piroctone olamine, zinc salt and a silicone. Anti-hair fall benefit includes preventing or reducing hair fall.

### Piroctone Olamine

Piroctone olamine (1-hydroxy-4-methyl-6-(2,4,4-trimethyl)-2-(1H)pyridinone,2-aminoethanol salt) is an ethanolamine salt of the hydroxamic acid derivative piroctone. Piroctone olamine (also known as piroctone ethanolamine; is the ethanolamine salt of the hydroxamic acid derivative piroctone. It is known as an antimycotic agent (brand name Octopirox®), and is used in antidandruff shampoos for the purpose of controlling the growth of Malassezia spp. It is believed to inhibit energy metabolism in mitochondria of pathogenic fungi by penetrating the fungal cell membrane and forming complexes with iron.

The piroctone olamine included in the hair care composition for use in the invention is preferably at a level of from 0.05 to 5%, more preferably from 0.1 to 2%, further more preferably from 0.1 to 1.5%, most preferably from 0.2 to 1% by weight of the total weight of the composition.

### Zinc Salt

The zinc salt included in the hair care composition for use in the invention may be selected from one or more of zinc acetate, zinc gluconate, zinc ammonium sulfate, zinc ascorbate, zinc bromide, zinc carbonate, zinc chloride, zinc citrate, zinc fluoride, zinc formate, zinc glycerophosphate, zinc iodide, zinc lactate, zinc nitrate, zinc oxalate, zinc phosphate, zinc salicylate, zinc sulfate, zinc succinate, zinc sulphite, and zinc tartrate. References herein to salts should be taken to mean the salt in any form including, for example, hydrate form.

It is preferred that the salt is selected from slightly soluble zinc salt, soluble zinc salt and mixtures thereof, most preferably the zinc salt is soluble. As used herein the term “soluble” means a salt that has a solubility in water at 20° C. and 1 atm of greater than 1 g salt/100 ml water. “Insoluble” means a salt that has a solubility in water at 20° C. and 1 atm of less than 0.01 g salt/100 ml water. Therefore “slightly soluble” means a salt that has a solubility in water at 20° C. and 1 atm of from 0.01 to 1 g/100 ml.

It is preferred the soluble zinc salt for use in the present invention comprises one or more of zinc acetate, zinc gluconate, zinc ammonium sulfate, zinc bromide, zinc chloride, zinc fluoride, zinc formate, zinc glycerophosphate, zinc iodide, zinc lactate, zinc nitrate, zinc salicylate, zinc sulfate.

It is more preferred the soluble zinc salt for use in the present invention comprises one or more of zinc chloride, zinc nitrate, zinc sulfate, and zinc gluconate, most preferably comprises or is zinc sulfate.

The amount of zinc salt included in the composition for use in the present invention is preferably such that the composition comprises at least 0.005% by weight of the composition, more preferably at least 0.01%, most preferably at least 0.015%. It is preferred that composition for use in the present invention comprises zinc salt at a level from 0.005% to 2%, more preferably 0.01% to 1.5%, most preferably 0.015% to 1% by weight based on the total weight of the composition.

### Silicone

The hair care composition for use according to the invention additionally comprises a silicone.

It is preferred that silicone suitable for use in the compositions of the invention is selected from the group comprising polyalkyl siloxanes, polyaryl siloxanes, polyalkylaryl siloxanes, polyether siloxane copolymers, or amino silicones or mixtures thereof. It is more preferred that the silicone is dimethicone, or dimethiconol, or a mixture thereof.

It is preferred that the total amount of silicone is 0.01 to 10 wt %, more preferably 0.1 to 5 wt % and most preferably 0.5 to 3 wt % by weight of the total weight of the composition.

### Hair Care Composition

The hair care composition for use according to the invention is suitable for topical application to the hair and scalp.

Suitable hair care compositions for use in the invention include rinse-off or leave-on hair and scalp care compositions such as shampoos, conditioners, creams, lotions, gels, serums, mousses or oils. Rinse-off hair and scalp care compositions such as shampoos and conditioners are preferred.

Compositions for use in the invention will generally include a cosmetically acceptable vehicle. The term “cosmetically acceptable” means that the vehicle is suitable for topical application to the skin, has good aesthetic properties, is compatible with the at least one biotin binding compound and the at least one precursor for biotin biosynthesis selected from pimelic acid and/or salts thereof and any other ingredients, and will not cause any safety or toxicity concerns.

The vehicle may comprise an aqueous phase, an oil phase, an alcohol, a silicone phase or a mixture thereof, and may be in the form of an emulsion. Emulsions can have a range of consistencies including thin lotions (which may also be suitable for spray or aerosol delivery), creamy lotions, light creams and heavy creams.

Compositions for use in the invention may also be formulated in a single-phase carrier such as a hydrophobic or hydrophilic liquid. Suitable hydrophobic liquid carriers include liquid polyorganosiloxanes, mineral oils, hydrogenated polyisobutene, polydecene, paraffins and isoparaffins of at least 10 carbon atoms, aliphatic or aromatic ester oils (such as isopropyl myristate, lauryl myristate, isopropyl palmitate, diisopropyl sebacate, diisopropyl adipate and C12 to C15 alkyl benzoates), polyglycol ethers (such as polyglycol butanol ethers) and mixtures thereof. Suitable hydrophilic liquid carriers include water, monohydric or polyhydric aliphatic alcohols having 2 to 8, preferably 2 or 3 carbon atoms (such as ethanol and isopropanol, oligoglycol ethers having 2 to 5 repeat units (such as dipropylene glycol) and mixtures thereof.

Liquid form compositions for use in the invention may be thickened, for example using one or more water soluble or colloidally water soluble polymeric thickening agents. Suitable water soluble or colloidally water soluble polymeric thickening agents include hydroxyethyl cellulose, methyl cellulose, hydroxypropyl methyl cellulose, polyquaternium-10, carrageenan, guar gum, hydroxypropyl guar gum, xanthan gum, polyvinylalcohol, acrylic acid/ethyl acrylate copolymers, carboxyvinyl polymers, cross-linked polyacrylate polymers and polyacrylamide polymers.

Preferred types of composition for use in the invention include shampoos, oils and lotions, more preferably shampoo, which are intended for topical application to the hair and scalp.

The pH of the composition for use according to the present invention is preferably equal to or higher than 4.0, more preferably in the range of 5.0 to 10.0.

Shampoo compositions for use in the invention are generally aqueous (i.e. they have water or an aqueous solution as their major component), and will suitably comprise from 50 to 98%, preferably from 60 to 90% water by weight of the total weight of the composition.

Shampoo compositions for use in the invention will typically comprise one or anionic surfactants such as sodium laureth sulfate, sodium oleyl succinate, ammonium lauryl sulfosuccinate, ammonium lauryl sulphate, sodium dodecylbenzene sulphonate, triethanolamine dodecylbenzene sulfonate, sodium cocoyl isethionate, sodium lauryl isethionate, sodium N-lauryl sarcosinate, sodium lauryl sulfate, sodium lauryl ether sulfate (n) EO, (where n ranges from 1 to 3), ammonium lauryl sulfate and ammonium lauryl ether sulfate (n) EO, (where n ranges from 1 to 3).

Mixtures of any of the above described materials may also be used.

The total amount of anionic surfactant in shampoo compositions for use in the invention generally ranges from 5 to 30%, preferably from 8 to 20% by weight of the total weight of the composition.

Shampoo compositions for use in the invention may also include co-surfactants such as nonionic surfactants, which can be included in an amount ranging from 0.5 to 8%, preferably from 2 to 5% (by weight based on the total weight of the composition) and/or amphoteric or zwitterionic surfactants, which can be included in an amount ranging from 0.5 to 8%, preferably from 1 to 4% by weight based on the total weight of the composition. Representative nonionic surfactants include alkanolamides such as cocamide monoethanolamide and cocamide monoisopropanolamide; alkyl polyglucosides such as cocoglucoside and lauryl glucoside; and acyl glucamides such as cocoyl methyl glucamide. Representive amphoteric surfactant includes cocamidopropyl betaine.

It is more preferred that the shampoo composition for use in the invention comprises sodium laureth sulfate and co-surfactant which is cocamidopropyl betaine.

Mixtures of any of the above described materials may also be used.

Shampoo compositions for use in the invention may also include one or more cationic polymers, which can be included in an amount ranging from 0.01 to 5%, preferably from 0.05 to 2% by weight based on the total weight of the composition. Representative cationic polymers include cationic polysaccharide polymers such as cationic cellulose derivatives and cationic guar gum derivatives such as guar hydroxypropyltrimethylammonium chloride.

Shampoo compositions for use in the invention may also include one or more suspending agents, which can be included in an amount ranging from 0.05 to 5%, preferably from 0.1 to 3% (by weight based on the total weight of the composition).

Representative suspending agents include polyacrylic acids, cross-linked polymers of acrylic acid, copolymers of acrylic acid with a hydrophobic monomer, copolymers of carboxylic acid-containing monomers and acrylic esters, cross-linked copolymers of acrylic acid and acrylate esters, heteropolysaccharide gums and crystalline long chain acyl derivatives such as ethylene glycol distearate.

Hair oils and lotions for use in the invention typically have an oil phase containing at one or more cosmetically acceptable fatty materials which may be liquid or solid at room temperature (25° C.). Lotions are typically aqueous emulsions having an aqueous phase in addition to the oil phase.

Suitable cosmetically acceptable fatty materials include naturally derived oils (such as sunflower oil, borage oil, soybean oil, castor oil, olive oil and almond oil); esters of monoalcohols or of polyols with monocarboxylic or polycarboxylic acids, at least one of the alcohols and/or acids comprising at least one hydrocarbon-based chain containing at least 6 carbon atoms (such as octyl palmitate, isopropyl myristate, isopropyl palmitate, isopropyl isostearate, hexyl laurate, isohexyl laurate, isohexyl palmitate, decyl oleate, isodecyl oleate, hexadecyl stearate, decyl stearate, dihexyldecyl adipate, lauryl lactate, myristyl lactate, cetyl lactate, oleyl stearate, oleyl oleate, oeyl myristate, lauryl acetate, cetyl propionate, isononyl isononanoate, propylene glycol dicaprate, diisopropyl adipate, dibutyl adipate, and oeyl adipate); ethers (such as dicapryl ether); fatty alcohols (such as cetyl alcohol, stearyl alcohol and behenyl alcohol); isoparaffins (such as isooctane, isododecane and isohexadecane); silicone oils (such as cyclomethicone, dimethicone and dimethiconol); hydrocarbon oils (such as mineral oil, petrolatum and polyisobutene); fatty acids containing from 8 to 30 carbon atoms, (such as stearic acid, lauric acid, palmitic acid and oleic acid); vegetable fats (such as cocoa butter, coconut oil, palm oil and shea butter); petroleum-based, natural and synthetic waxes (such as lanolin wax, beeswax, carnauba wax, candelilla wax, paraffin wax, lignite wax, microcrystalline waxes, ceresin, ozokerite, and polyethylene waxes); hydrogenated oils which are solid at 25° C. (such as hydrogenated castor oil, hydrogenated jojoba oil, hydrogenated palm oil, hydrogenated tallow and hydrogenated coconut oil);and fatty esters that are solid at 25° C. (such as C20-40 alkyl stearate).

The aqueous phase of lotions for use in the invention may also include one or more organic liquids that are miscible with water at room temperature (25° C.). Exemplary water-miscible organic liquids include monohydric and polyhydric alcohols and derivatives thereof such as C2-C6 alkanols (such as ethanol and isopropanol); C2-C10 glycols and polyols (such as glycerol, propylene glycol, butylene glycol, pentylene glycol, hexylene glycol, caprylyl glycol, dipropylene glycol, and diethylene glycol); C3-C16 glycol ethers (such as mono-, di-, or tripropylene glycol (C1-C4) alkyl ethers and mono-, di-, or triethylene glycol (C1-C4) alkyl ethers) and polyethylene glycol having 2 to 12 oxyethylene units.

Lotions for use in the invention may also include surface active ingredients, such as emulsifiers and solubilizers, to enable two or more immiscible components to be combined homogeneously and to help stabilize the composition. Emulsifiers that may be used to form O/W or W/O emulsions include sorbitan oleate, sorbitan sesquioleate, sorbitan isostearate, sorbitan trioleate, PEG-20 sorbitan isostearate, polyglyceryl-3-diisostearate, polyglycerol esters of oleic/isostearic acid, polyglyceryl-6 hexaricinolate, polyglyceryl-4-oleate, polyglyceryl-4 oleate/PEG-8 propylene glycol cocoate, polyglyceryl-2 dipolyhydroxystearate, PEG-30 dipolyhydroxystearate, oleamide DEA, TEA myristate, TEA stearate, magnesium stearate, sodium stearate, potassium laurate, potassium ricinoleate, sodium cocoate, sodium tallowate, potassium castorate, sodium oleate, cetyl phosphate, diethanolamine cetyl phosphate, potassium cetyl phosphate, sodium glyceryl oleate phosphate, dimethicone copolyol, cetyl dimethicone copolyol, octyldimethicone ethoxyglucoside copolyol, dimethicone copolyol crosspolymer and laurylmethicone copolyol.

Combinations of any of the above described materials or product forms may also be used.

Compositions for use in the invention (as described above) may include additional actives for improving the physical and/or aesthetic characteristics of the scalp and/or the hair. Examples include amino acids, vitamins, minerals and/or antioxidants, emollients, humectants, sunscreens, anti-irritants, exfoliating agents, botanical extracts (such as pomegranate, white birch, green tea, chamomile and licorice extracts) and mixtures thereof.

Compositions for use in the invention (as described above) may include additional functional ingredients for improving the physical and/or aesthetic characteristics of the composition per se. Examples include inorganic pigments (such as titanium oxide, zirconium oxide, cerium oxide, zinc oxide, iron oxide, chromium oxide, manganese violet, ultramarine blue, chromium hydrate and ferric blue); organic pigments (such as carbon black and the organic lakes of barium, strontium, calcium or aluminium); pearlescent agents (such as mica coated with titanium oxide and/or iron oxide); dyes, preservatives (such as disodium EDTA, benzyl alcohol, methylparaben, phenoxyethanol, propylparaben, ethylparaben, butylparaben and isobutylparaben); pH adjusters and fragrances (such as essential oils, flower oils, natural extracts from resins, gums, balsams, beans, mosses and other plants, as well as synthetic aromatic materials).

Mixtures of any of the above described materials may also be used.

### Method

The invention provides for a method of preventing or reducing hair fall in an individual in need thereof, comprising topically applying a hair care composition comprising piroctone , a zinc salt and a silicone to the hair and/or scalp of the individual, in an amount effective to reduce shedding of hair club fibres from hair follicles of the individual, and/or reduce hair breakage of the individual. Preferably, the present invention provides for a method of preventing or reducing hair fall in an individual in need thereof, comprising topically applying a hair care composition comprising piroctone, a zinc salt and a silicone to the hair and/or scalp of the individual, in an amount effective to reduce shedding of hair club fibres from hair follicles of the individual. The method is preferably non-therapeutic in nature, more preferably cosmetic in nature.

The composition is suitably applied to the hair and scalp and massaged into the surface of the scalp. Generally, an amount corresponding to about 1 to 15 ml of the composition per application is applied uniformly over the area of treatment daily or at least once a week over a time interval of at least 7 (seven) days, preferably at least 28 days, more preferably at least 30 (thirty) days.

The present invention provides a hair care composition comprises piroctone olamine, a zinc salt and a silicone for use in preventing or reducing shedding of hair club fibres from hair follicles, and/or preventing or reducing hair breakage, thereby preventing or reducing hair fall.

The present invention provides a hair care composition for use in preventing or reducing shedding of hair club fibres from hair follicles, and/or preventing or reducing hair breakage, thereby preventing or reducing hair fall; wherein the hair care composition comprises: piroctone olamine, wherein the hair care composition provides a greater anti-hair fall benefit in comparison to a hair care composition that does not contain piroctone olamine. Anti-hair fall benefit includes preventing or reducing hair fall.

The present invention provides a hair care composition for use in preventing or reducing shedding of hair club fibres from hair follicles, and/or preventing or reducing hair breakage, thereby preventing or reducing hair fall; wherein the hair care composition comprises: piroctone olamine; a zinc salt; and a silicone; wherein the hair care composition provides a greater anti-hair fall benefit in comparison to a hair care composition that does not contain the combination of piroctone olamine, a zinc salt and a silicone. Anti-hair fall benefit includes preventing or reducing hair fall.

The invention will be further illustrated by the following, non-limiting Examples.

### EXAMPLES

### Example 1

This example demonstrates the anti-hair fall activity of compositions.

The following shampoo compositions were prepared as shown in Table 1:

TABLE 1

| | Composition 1 | Composition A |
|---|---|---|
| Ingredient | (wt % as active) | (wt % as active) |
| Sodium laureth sulfate | 13.00 | 13.00 |
| Cocamidopropyl betaine | 1.30 | 1.30 |
| Guar hydroxypropyltrimonium | 0.19 | 0.19 |
| chloride¹ | | |
| Carbomer | 0.35 | 0.35 |
| Piroctone olamine | 0.50 | — |
| DC1788: Dimethiconol | 1.50 | — |
| DC7128: Dimethicone | 1.00 | — |
| Zinc Sulfate | 0.018 | — |
| Preservative | 0.8 | 0.8 |
| Fragrance | 0.80 | 0.80 |
| Citric acid | 0.154 | 0.154 |
| Sodium hydroxide | 0.16 | 0.16 |
| Sodium chloride | 1.20 | 1.20 |
| Water | To 100 | To 100 |

¹The cationic guar used had a molecular weight of about 1.35 million Dalton and a degree of substitution of 0.18 sourced from Lamberti.

### Clinial Study Method

Two shampoos, one active composition within the scope of the present invention (Composition 1) and a placebo composition out of the scope (Composition A), were applied in a double blind clinical study to determine the efficacy of the combination to reduce hair shedding and total hair fall (including hair shedding and hair breakage) after 4 weeks of product usage.

124 female subjects volunteered for the study and 122 subjects completed the study. The subjects had self-perceived hair loss and self-perceived dandruff and with hair length at least 8 cm but no longer than the base of shoulder.

Run-in phase:

All qualified subjects after initial screening were given a non-AD beauty shampoo to wash hair three times per week for 2 weeks at home.

Baseline test:

After 2 weeks of run-in phase, subjects came back to the study center and had their hair washed by a trained staff, using the run-in shampoo following salon whole head wash procedure. The fallen hair strands during washing, drying and combing stage were collected and put in the clean plastic bag. The collected hair strands were visually inspected and classified as either shed hair (with a typical club end of an exogen hair) or broken hair by trained staff. The number of shed hair and hair fall (shed and broken hair) at this visit were recorded as baseline.

Product test:

The subjects were randomly split into two groups and allocated with active shampoo (Composition 1) or placebo shampoo (Composition A). During 4 weeks of test phase, subjects were asked to wash hair 3 times per week and total 12 washes in the test phase. The last 3 washes at week 4 were done at study center by trained staff. The fallen hair strands during washing, drying and combing were again collected. The collected hair strands were visually inspected and classified as either shed hair or broken hair by trained staff. The average hair count number of 3 washes in week 4 was recorded.

Analysis method:

Repeated analysis of covariance (ANCOVA) was used to analyse the shed hair and total hair fall (shed hair and broken hair) data, separately. Baseline shed hair or baseline total hair fall (she hair and broken hair) data was used as covariate.

### Results

The efficacy of the composition of the present invention on reducing hair shedding is shown in Table 2:

| | | | | | Placebo |
|---|---|---|---|---|---|
| Hair | Placebo (Composition A) | | Active (Composition 1) | | vs. |
| shedding | (n = 62) | | (n = 60) | | Active |
| counted | Mean | Std | Mean | Std | p value |
| Baseline | 187.03 | 57.93 | 184.88 | 62.96 | — |
| Week 4 | 131.40 | 29.30 | 116.34 | 40.83 | 0.0185 |

The data in Table 2 illustrates that the treatment with the composition within the scope of the present invention (Composition 1) can effectively prevent or reduce hair shedding, and consequently prevent or reduce hair fall, comparing with the composition out of the scope of the present invention (Composition A).

The efficacy of the composition of the present invention on reducing total hair fall (hair shedding and hair breakage) is shown in Table 3:

| | | | | | Placebo |
|---|---|---|---|---|---|
| Total | Placebo (Composition A) | | Active (Composition 1) | | vs. |
| hairfall | (n = 62) | | (n = 60) | | Active |
| counted | Mean | Std | Mean | Std | p value |
| Baseline | 193.85 | 59.56 | 191.52 | 62.91 | — |
| Week 4 | 135.61 | 29.51 | 118.62 | 40.83 | 0.0088 |

The data in Table 3 illustrates that the treatment with the composition within the scope of the present invention (Composition 1) can effectively prevent or reduce hair fall which include the hair shedding and hair breakage, comparing with the composition out of the scope of the present invention (Composition A).

1-11. (canceled)

12. A method of preventing or reducing hair fall in an individual in need thereof, comprising topically applying a hair care composition comprising piroctone olamine, a zinc salt and a silicone to the hair and/or scalp of the individual, in an amount effective to reduce shedding of hair club fibres from hair follicles of the individual, and/or reduce hair breakage of the individual.

13-14. (canceled)

15. The method of claim 12, wherein the hair composition is a rinse-off composition.

16. The method of claim 12, wherein the hair composition is a shampoo.

17. The method of claim 12, wherein the hair composition comprises 0.05% to 5% by weight of the piroctone olamine based on the total weight of the composition.

18. The method of claim 12, wherein the zinc salt is at least one of zinc acetate, zinc gluconate, zinc ammonium sulfate, zinc ascorbate, zinc bromide, zinc carbonate, zinc chloride, zinc citrate, zinc fluoride, zinc formate, zinc glycerophosphate, zinc iodide, zinc lactate, zinc nitrate, zinc oxalate, zinc phosphate, zinc salicylate, zinc sulfate, zinc succinate, zinc sulphite, and zinc tartrate.

19. The method of claim 12, wherein the zinc salt is soluble.

20. The method of claim 19, wherein the zinc salt is chosen from zinc chloride, zinc nitrate, zinc sulfate, zinc gluconate, or a combinations thereof.

21. The method of claim 19, wherein the hair composition comprises 0.005% to 2% by weight of the zinc salt based on the total weight of the composition.

22. The method of claim 12, wherein the silicone is at least one of a polyalkyl siloxane, a polyaryl siloxane, a polyalkylaryl siloxane, a polyether siloxane copolymer, and an amino silicone.

23. The method of claim 12, wherein the silicone is chosen from dimethicone, dimethiconol, or a combination thereof.

24. The method of claim 12, wherein the hair composition comprises 0.01% to 10% by weight of the silicone based on the total weight of the composition.
